(19) United States
(12) Patent Application Publication
(10) Pub. No.: US 2025/0387308 A1
(43) Pub. Date: Dec. 25, 2025

(54) Deodorizing Formula and Methods of Production of the Same

(71) Applicant: David John Whelan, Minneapolis, MN (US)

(72) Inventor: David John Whelan, Minneapolis, MN (US)

(21) Appl. No.: 19/244,801

(22) Filed: Jun. 20, 2025

Related U.S. Application Data
(60) Provisional application No. 63/661,802, filed on Jun. 19, 2024.

(51) Int. Cl. A61K 8/19; A61Q 15/00

(52) U.S. Cl. CPC A61K 8/19; A61Q 15/00; A61K 2800/413; A61K 2800/49; A61K 2800/5922; A61K 2800/624

(57) ABSTRACT

A deodorizing formula including water, a first solute selected from sodium or potassium percarbonate, a second solute selected from sodium or potassium carbonate, and silver nano-particles wherein said silver particles may be selected from silver colloid particles, silver colloid particles coated with polyvinylpyrrolidone (PVP), and silver colloid particles comprising coated with a natural polymer. The first solute releases oxygen upon decomposition in water, while the second solute stabilizes the released oxygen. The silver nanoparticles penetrate skin pores to eliminate odor-causing bacteria and neutralize odor compounds. The method includes sequential mixing of components, aging the solution, and packaging into sprayable or topical forms for immediate and long-lasting deodorizing effects.

### FIELD OF THE INVENTION

The present invention relates generally to a formula. More specifically, the present invention is a formula for deodorizing skin.

### BACKGROUND OF THE INVENTION

Body odor, particularly under the arms, feet, and groin area, results primarily from microbial activity on the skin. Bacteria such as Staphylococcus, Corynebacterium, and Propionibacterium metabolize organic compounds found in sweat and sebum, producing a range of malodorous compounds including isovaleric acid, 3-methyl-2-hexenoic acid (3M2H), hydrogen sulfide, methyl mercaptan, cadaverine, and putrescine. These metabolites are responsible for the characteristic unpleasant smell of body odor. Traditional deodorants and antiperspirants often rely on masking agents, alcohol-based bactericides, or aluminum salts to reduce odor or sweat. However, such solutions may only provide temporary effects, cause irritation, clog pores, or raise concerns over long-term safety.

Accordingly, there remains a significant need for a topical deodorizing solution that is safe, non-irritating, fast-acting, and capable of neutralizing odor-causing compounds at the source—both chemically and microbiologically—without blocking sweat glands or relying heavily on synthetic fragrances.

Prior art in the field of deodorizing compositions includes a variety of topical sprays, roll-ons, and creams incorporating antimicrobial agents such as triclosan, essential oils, or alcohol; oxidizing agents such as hydrogen peroxide; and adsorbents such as baking soda or activated charcoal. Some compositions use silver ions for antimicrobial action, but often lack particle stability or penetration efficacy. Many commercial formulations also rely on aluminum-based compounds to block sweat ducts or incorporate strong synthetic fragrances to mask odor, which may cause irritation or allergic reactions in sensitive individuals.

One limitation of existing formulations is the insufficient stabilization of reactive ingredients such as percarbonates or silver colloids in aqueous environments. For instance, sodium percarbonate readily decomposes in water, releasing oxygen, but without a stabilizing component, the oxidation is transient and ineffective for long-term odor control. Furthermore, current products often do not provide a formulation with nano-scale silver particles small enough (2-100 nm) to penetrate into skin pores (typically 250-500 nm), limiting their reach and efficacy against intradermal bacteria.

The present invention addresses these shortcomings through a formulation comprising water, an oxidant (sodium percarbonate or potassium percarbonate), a stabilizer (sodium or potassium carbonate), and nano-sized silver colloids coated with either polyvinyl pyrrolidone (PVP) or a natural polymer. This composition is designed to deliver immediate deodorizing action by chemically neutralizing odor-causing metabolites and biologically eliminating the bacteria responsible for producing them. The percarbonate releases oxygen into the solution, enabling oxidation of odorants and bacterial disruption. The carbonate component helps retain the oxygen in solution, enhancing longevity and stability. The nano silver colloid particles act as a skin-penetrating antibacterial agent, reaching deep into the pores where microbes reside.

This combination offers several advantages over the prior art: (1) rapid elimination of body odor; (2) long-lasting protection for up to 12 hours; (3) effective bacterial neutralization both on the skin's surface and within pores; and (4) minimal irritation due to the absence of alcohol, aluminum, or synthetic preservatives. Furthermore, scented variations of the formula may include perfume oils, solubilizers, emulsifiers, and surfactants for enhanced sensory appeal, product stability, and user experience.

The present invention thus fills a longstanding need in the personal hygiene industry for a safe, effective, and science-backed deodorizing solution, offering substantial improvements over conventional deodorants and antiperspirants.

### SUMMARY OF THE INVENTION

A deodorizing formula for deodorizing skin and a method of production of the same. The deodorizing formula includes a plurality of solutes, a quantity of water, and a quantity of silver particles. In preferred embodiments, the plurality of solutes includes a first solute selected from sodium percarbonate and potassium percarbonate (1-10% by volume), and a second solute selected from sodium carbonate and potassium carbonate (1-10% by volume). These components interact with water to release and stabilize oxygen within the solution, enhancing deodorizing and antibacterial activity.

The formula further includes silver colloid particles, preferably coated with polyvinylpyrrolidone (PVP) or a natural polymer, having particle sizes ranging from 2 nm to 100 nm. These nano-sized particles are sufficiently small to penetrate human skin pores (250-500 nm) and eliminate odor-causing bacteria such as Staphylococcus, Corynebacterium, and Propionibacterium. In addition to eradicating the bacterial source, the silver particles also neutralize associated odor compounds including isovaleric acid, 3-methyl-2-hexenoic acid, hydrogen sulfide, and other metabolic byproducts. Furthermore, the addition of a natural polymer prevents the silver colloid particles from washing off the skin.

The method of production comprises: (1) forming a solution of the first solute and water to generate oxygen through decomposition of percarbonate; (2) adding the second solute to stabilize the oxygen in solution; (3) incorporating the silver particles; and (4) in some embodiments, adding cosmetic additives such as perfume oils, solubilizers, emulsifiers, and surfactants. These cosmetic additives enhance fragrance, stability, and cleansing properties. The formula is then formed into various compositions such as liquid sprays, gels, lotions, or creams.

### BRIEF DESCRIPTION OF THE DRAWINGS

FIG. 1 is a composition table of the present invention.

FIG. 2 is a table of the quantity of a first solute of the present invention.

FIG. 3 is a table of the quantity of a second solute of the present invention.

FIG. 4 is a table of the quantity of silver particles of the present invention.

FIG. 5 is a table of the silver colloid particles comprising a quantity of natural polymer of the present invention.

FIG. 6 is a diagram of the deodorizing formula applied to the skin of a user.

FIG. 7 is a process diagram of the process to manufacture the deodorizing formula.

FIG. 8 is a process diagram of the process to manufacture the deodorizing formula showing a supplemental fourth step.

FIG. 9 is a table of the compositions of the present invention.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a deodorizing formula 1 and a method 20 of producing the same, embodiments of the present disclosure are not limited to use only in this context. In the context of the present invention, deodorizing formula 1 may also be referred to as deodorizing composition, herein.

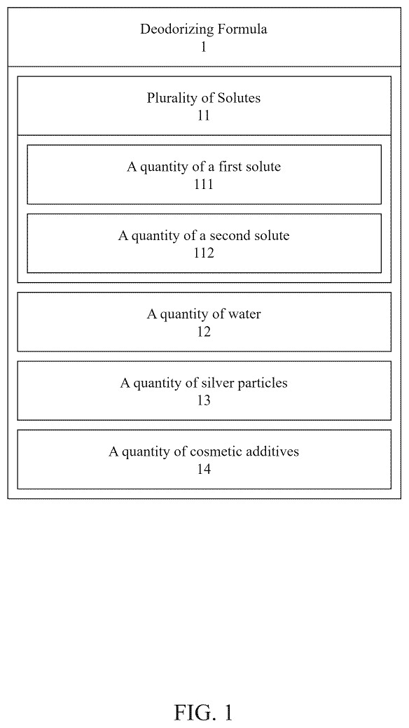

As shown in FIGS. 1-8, the present invention is a deodorizing formula 1 for deodorizing skin and a method 20 of production of the same. As shown in FIG. 1, the deodorizing formula 1 is composed of a plurality of solutes 11, a quantity of water 12, and a quantity of silver particles 13. In the context of the present invention, the plurality of solutes comprises a quantity of a first solute 111 and a quantity of a second solute 112. Furthermore, in some embodiments of the present invention, the deodorizing formula 1 comprises a quantity of cosmetic additives 14.

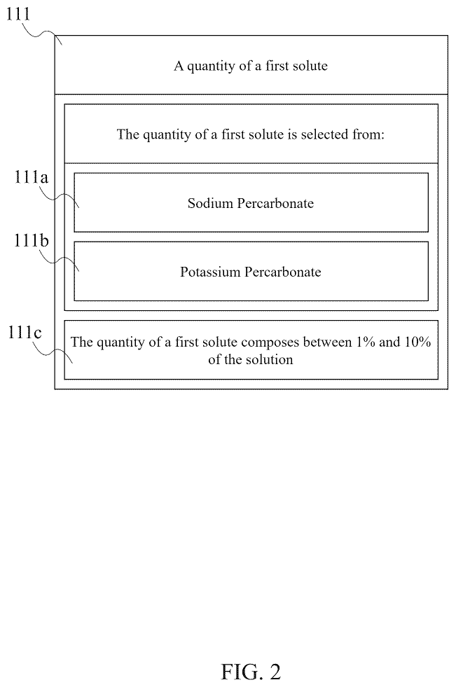

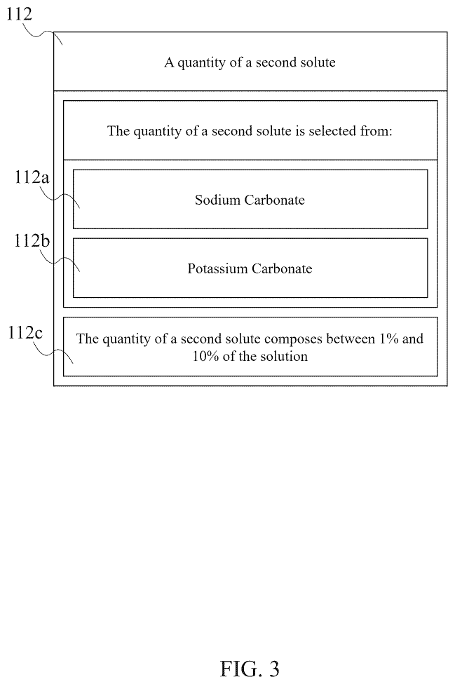

In the preferred embodiment of the present invention, as shown in FIG. 2, the quantity of a first solute 111 is selected from sodium percarbonate 111a and potassium percarbonate 111b. Additionally, within the preferred embodiments of the present invention, the quantity of a first solute 111 composes between 1%-10% by volume 111c of the deodorizing formula 1. Moreover, as shown in FIG. 3, in the preferred embodiment of the present invention, the quantity of a second solute 112 is selected from sodium carbonate 112a and potassium carbonate 112b. Additionally, in the preferred embodiment of the present invention, the quantity of a second solute composes between 1%-10% by volume 112c of the deodorizing formula 1.

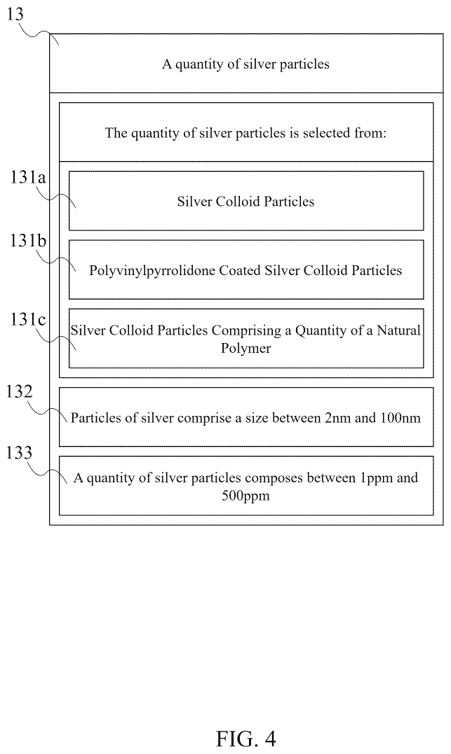

Referring to FIG. 4, in the preferred embodiment of the present invention, the quantity of silver particles 13 is selected from a quantity of silver colloid particles 131a, a quantity of polyvinylpyrrolidone (PVP) coated silver colloid particles 131b, and a quantity of silver colloid particles comprising a quantity of a natural polymer 131c. In the preferred embodiment of the present invention, each particle of the quantity of silver particles 13 comprises a size between 2 nm and 100 nm in diameter 132. Additionally, within the preferred embodiment of the present invention, the quantity of silver particles 13 composes between 1 ppm and 500 ppm 133 of the deodorizing formula 1.

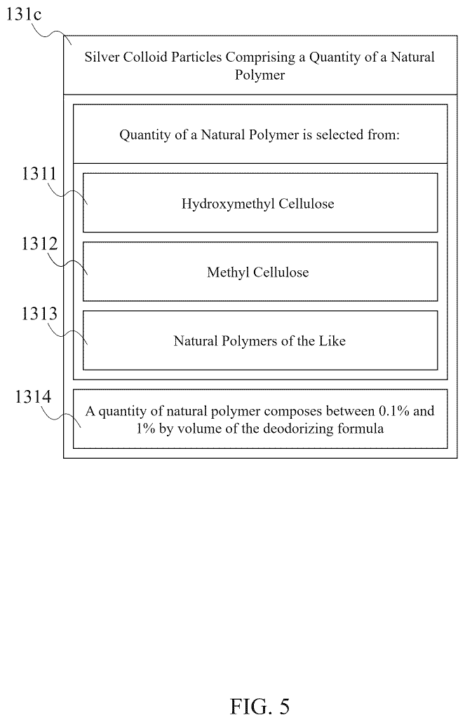

In the context of the present invention, as shown in FIG. 5, in such embodiments wherein the quantity of silver particles 13 is a quantity of silver colloid particles comprising a quantity of natural polymer 131c, the quantity of natural polymer is selected from hydroxymethyl cellulose 1311, methyl cellulose 1312, and natural polymers of the like 1313. Further, in such embodiments comprising a quantity of natural polymer, said natural polymer composes between 0.1% and 1% of the deodorizing formula 1 by volume 1314. Moreover, in such embodiments of the present invention wherein the quantity of silver particles 13 is a quantity of silver colloid particles comprising a quantity of natural polymer 131c, the quantity of natural polymer prevents the silver particles from washing off (i.e. releasing from) the skin of a user. Additionally, the addition of the quantity of natural polymer has been found to prevent the formation of foam within the deodorizing formula 1, thus acting as an anti-foaming agent.

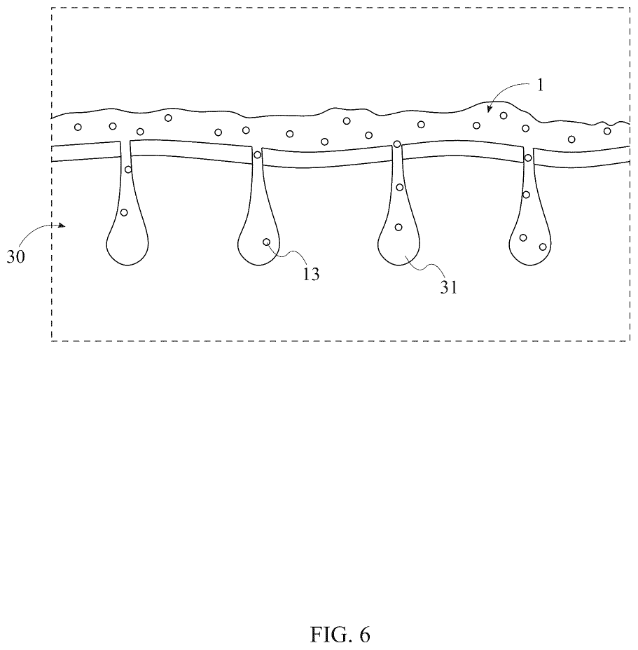

Within the context of the present invention, as shown in FIG. 6, the size of the particles of the quantity of silver particles 13 enables the silver particles 13 to penetrate pores 31 of human skin 30—as human pores 31 are between 250-500 nm in diameter. Additionally, in the context of the present invention, the quantity of silver particles 13 kill bacterial organisms within the dermis of human skin 30 including Staphylococcus, Corynebacterium and Propionibacterium—and their corresponding metabolic waste products including thioalcohols and fatty acids that contribute to body odor, thereby deodorizing the skin 30. Furthermore, the quantity of silver particles 13 eliminates odors produced by chemicals including isovaleric acid, 3-methyl-2-hexenoic acid (3M2H), 3-hydroxy-3methylhexanoic acid (HMHA), 3-methyl-3-sulfanylhexan-1-ol (3M3SH), hydrogen sulfide (H2S), methyl mercaptan (from sulfur amino acids), cadaverine (from lysine), putrescine (from ornithine), and ammonia.

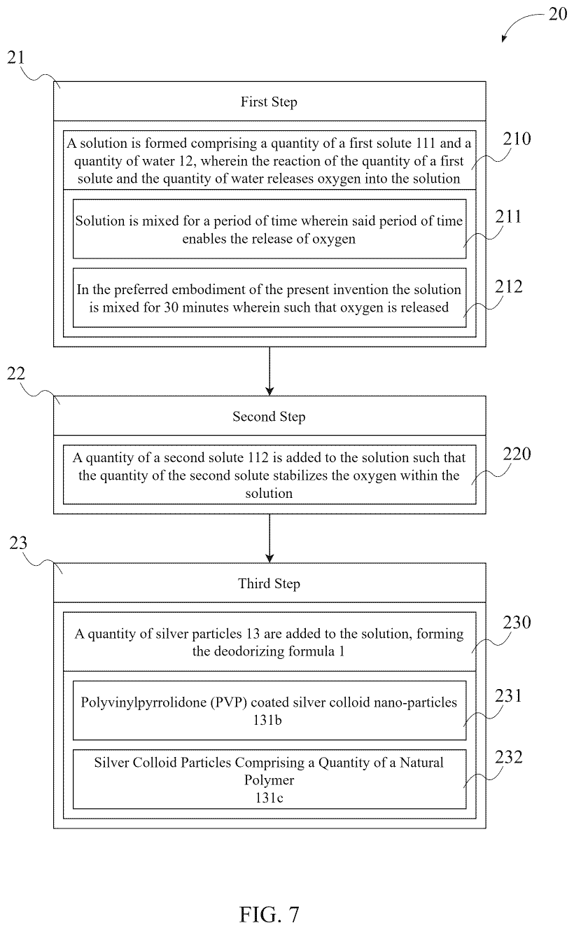

Referring to FIG. 7, in the preferred embodiment of the present invention, the deodorizing formula 1 is produced through a process 20 comprising a first step 21 wherein a solution is formed comprising the quantity of the first solute 111 and the quantity of the water 12 such that the reaction between the quantity of the first solute 111 and the quantity of water 12 releases oxygen into the solution. Further, in the preferred embodiment of the present invention, the solution is mixed for a period of time wherein said period of time enables the release of oxygen into the solution 211. Furthermore, in the preferred embodiment of the present invention, the solution is mixed for 30 minutes 212. In the context of the present invention, upon being added to a quantity of water 12, sodium percarbonate 111a decomposes, producing sodium carbonate and hydrogen peroxide—wherein said hydrogen peroxide further decomposes, releasing oxygen into the solution. Similarly, in the context of the present invention, upon being added to a quantity of water 12, potassium percarbonate 111b decomposes, producing potassium carbonate and hydrogen peroxide—wherein said hydrogen peroxide further decomposes, releasing oxygen into the solution.

Further referring to FIG. 7, in the preferred embodiment of the present invention, the process 20 of producing the deodorizing formula 1 comprises a second step 22 wherein the quantity of a second solute 112 is added to the solution such that the quantity of a second solute 112 stabilizes oxygen within the solution 220. In the context of the present invention, upon the release of oxygen into the solution, a quantity of a second solute 112 selected from a quantity of sodium carbonate 112a and a quantity of potassium carbonate 112b is added to the solution, thereby stabilizing the oxygen within the solution.

Moreover, as shown in FIG. 7, in the preferred embodiment of the present invention, the process 20 of producing the deodorizing formula 1 further comprises a third step 23 wherein a quantity of silver particles 13 are added to the solution 230, thus producing the deodorizing formula 1. In some embodiments of the present invention, a quantity of PVP coated silver colloid nano-particles 131b are added to the solution 231, producing the deodorizing formula 1. In alternate embodiments, a quantity of silver colloid particles comprising a quantity of natural polymer 131c is added to the solution 232, producing the deodorizing formula 1.

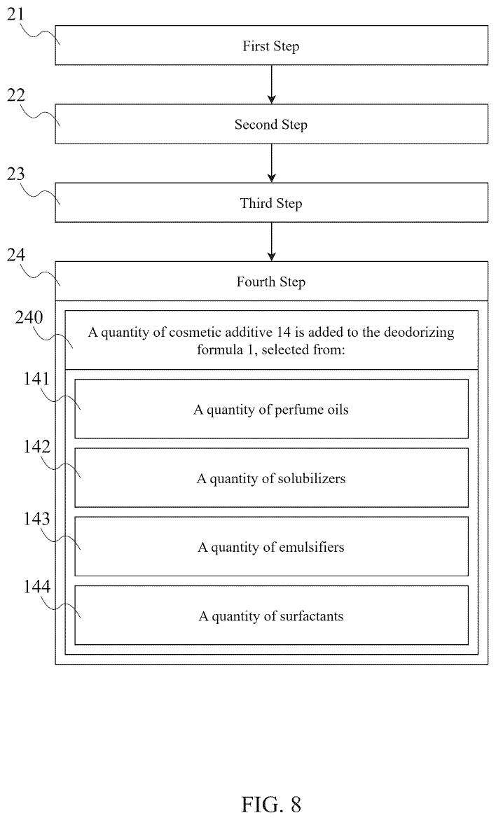

As shown in FIG. 8, in some embodiments of the present invention, the process 20 of producing the deodorizing formula 1 further comprises a fourth step 24 wherein a quantity of cosmetic additive 14 is added 240 to the deodorizing formula 1 wherein said cosmetic additive 14 is selected from a quantity of perfume oils 141, a quantity of solubilizers 142, a quantity of emulsifiers 143, and a quantity of surfactants 144. In the context of the present invention, perfume oils are understood to be aromatic compounds added to the deodorizing formula to impart a pleasant scent. In the context of the present invention, solubilizers are understood to be agents that enables the dissolution of otherwise insoluble ingredients. In the context of the present invention, emulsifiers are understood to be substances that stabilize mixtures by reducing the surface tension between oil and water. Lastly, in the context of the present invention, surfactants are understood to be surface-active agents that behave as at least one of cleansers, foaming agents, and deodorants.

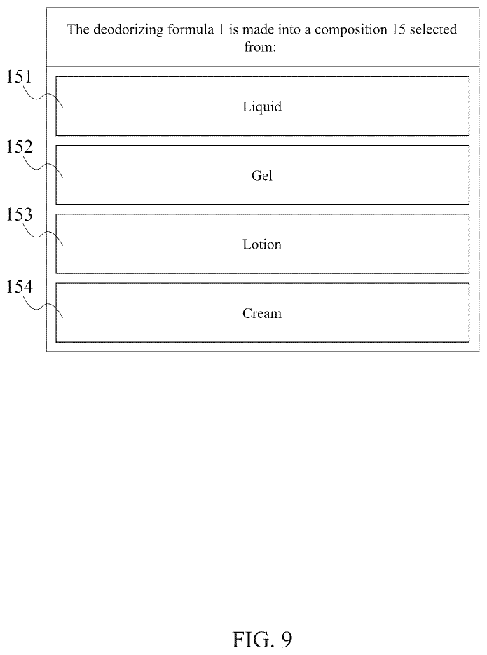

As shown in FIG. 9, in the preferred embodiment of the present invention, the deodorizing formula 1 is formed into a composition 15 selected from a liquid 151, a gel 152, a lotion 153, and a cream 154. Moreover, in the preferred embodiment of the present invention, the deodorizing formula 1 is formed into a composition 15, wherein said composition 15 is bottled after at least 48 hours. In the preferred embodiment of the present invention, the deodorizing formula 1 is contained within a spray bottle wherein said spray bottle enables a user to spray the deodorizing formula 1 onto the skin 30 of the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

Although the invention has been explained in relation to its preferred embodiment, it is to be understood that many other possible modifications and variations can be made without departing from the spirit and scope of the invention.

What is claimed is:

1. A deodorizing formula comprising:
a quantity of a first solute;
a quantity of a second solute;
a quantity of water; and
a quantity of silver particles;
wherein:
the quantity of a first solute is selected from:
a quantity of sodium percarbonate; and
a quantity of potassium percarbonate; and
the quantity of a second solute is selected from:
a quantity of sodium carbonate; and
a quantity of potassium carbonate.

2. The deodorizing formula, as claimed in claim 1, wherein the quantity of a first solute composes between 1% and 10% by volume of the deodorizing formula.

3. The deodorizing formula, as claimed in claim 2, wherein the quantity of a second solute composes between 1% and 10% by volume of the deodorizing formula.

4. The deodorizing formula, as claimed in claim 3, wherein the quantity of silver particles composes between 1 ppm and 500 ppm of the deodorizing formula.

5. The deodorizing formula, as claimed in claim 4, wherein each of the particles of silver of the quantity of silver particles are between 2 nm and 100 n in diameter.

6. The deodorizing formula, as claimed in claim 1, wherein the quantity of silver particles is a quantity of silver colloid particles between 2 nm and 100 n in diameter.

7. The deodorizing formula, as claimed in claim 1, wherein the quantity of silver particles is selected from:
a quantity of polyvinylpyrrolidone coated silver colloid particles; and
a quantity of silver colloid particles comprising a quantity of natural polymer such that the quantity of natural polymer composes between 0.1% and 1% of the deodorizing formula by weight, wherein said quantity of natural polymer is selected from:
hydroxymethyl cellulose; and
methyl cellulose.

8. The deodorizing formula, as claimed in claim 1, wherein:
the quantity of a first solute is combined with the quantity of water, producing a solution, such that oxygen is released into the solution;
the quantity of a second solute is added to the solution, stabilizing the oxygen within the solution; and
the quantity of silver particles being added to the solution, thus composing the deodorizing formula.

9. The deodorizing formula, as claimed in claim 1, further comprising a quantity of cosmetic additive, wherein said quantity of cosmetic additive is selected from:
a quantity of perfume oil;
a quantity of solubilizer;
a quantity of emulsifier; and
a quantity of surfactant.

10. The deodorizing formula, as claimed in claim 8, further comprising a quantity of cosmetic additive, wherein said quantity of cosmetic additive is selected from:
a quantity of perfume oil;
a quantity of solubilizer;
a quantity of emulsifier; and
a quantity of surfactant.

11. A composition formed from the deodorizing formula of claim 1, wherein the deodorizing formula is formed into a liquid.

12. A composition formed from the deodorizing formula of claim 1, wherein the deodorizing formula is formed into a gel.

13. A composition formed from the deodorizing formula of claim 1, wherein the deodorizing formula is formed into a lotion.

14. A composition formed from the deodorizing formula of claim 1, wherein the deodorizing formula is formed into a cream.

15. A deodorizing formula comprising:
a quantity of a first solute;
a quantity of a second solute;
a quantity of water; and
a quantity of silver particles selected from:
a quantity of polyvinylpyrrolidone coated silver colloid particles; and
a quantity of silver colloid particles comprising a quantity of natural polymer wherein said quantity of natural polymer is selected from:
hydroxymethyl cellulose; and
methyl cellulose;
wherein:
the quantity of a first solute is selected from:
a quantity of sodium percarbonate; and
a quantity of potassium percarbonate;
the quantity of a second solute is selected from:
a quantity of sodium carbonate; and
a quantity of potassium carbonate;
the quantity of a first solute composes between 1% and 10% by volume of the deodorizing formula;
the quantity of a second solute composes between 1% and 10% by volume of the deodorizing formula; and
the quantity of silver particles such that each silver particle is between 2 nm and 100 n in diameter.

16. The deodorizing formula, as claimed in claim 15, wherein the quantity of silver particles composes between 1 ppm and 500 ppm of the deodorizing formula.

17. The deodorizing formula, as claimed in claim 16, further comprising a quantity of cosmetic additive, wherein said quantity of cosmetic additive is selected from:
a quantity of perfume oil;
a quantity of solubilizer;
a quantity of emulsifier; and
a quantity of surfactant.

18. The deodorizing formula, as claimed in claim 16, wherein:
the quantity of a first solute is combined with the quantity of water, producing a solution, such that oxygen is released into the solution;
the quantity of a second solute is added to the solution, stabilizing the oxygen within the solution;
the quantity of silver particles being added to the solution, thus composing the deodorizing formula; and
the deodorizing formula is formed into a composition selected from:
a liquid;
a gel;
a lotion; and
a cream.

19. A deodorizing formula comprising:
a quantity of a first solute;
a quantity of a second solute;
a quantity of water;
a quantity of polyvinylpyrrolidone coated silver colloid particles; and
a quantity of a cosmetic additive;
wherein:
the quantity of a first solute is selected from:
a quantity of sodium percarbonate; and
a quantity of potassium percarbonate;
the quantity of a second solute is selected from:
a quantity of sodium carbonate; and
a quantity of potassium carbonate;
the quantity of cosmetic additive is selected from:
a quantity of perfume oil;
a quantity of solubilizer;
a quantity of emulsifier; and
a quantity of surfactant
the quantity of a first solute composes between 1% and 10% of the deodorizing formula;
the quantity of a second solute composes between 1% and 10% of the deodorizing formula; and
the quantity of polyvinylpyrrolidone coated silver colloid particles composes between 1 ppm and 500 ppm of the deodorizing formula.

20. A composition formed from the deodorizing formula of claim 19, wherein the deodorizing formula is formed into a composition selected from:
a liquid;
a gel;
a lotion; and
a cream.
